(19) United States
(12) Patent Application Publication
(10) Pub. No.: US 2005/0268912 A1
(43) Pub. Date: Dec. 8, 2005

(54) System and method for automated titration of continuous positive airway pressure

(76) Inventors: Robert G. Norman, New Windsor, NY (US); David M. Rapoport, New York, NY (US)

Correspondence Address: FAY KAPLUN & MARCIN, LLP, 15O BROADWAY, SUITE 702, NEW YORK, NY, 10038, US

(21) Appl. No.: 10/862,067

(22) Filed: Jun. 4, 2004

(51) Int. Cl. A62B 7/00; A61M 16/00

(52) U.S. Cl. 128/204.23; 128/204.18; 128/204.21

(57) ABSTRACT

Described is a method and system for automated titration of CPAP. The system may include an air pressure supply providing air pressure to a patient's airways and a sensor detecting input data corresponding to a patient's breathing patterns of a plurality of breaths. The system also includes a titration device which receives and analyzes the input data to determine existence of breathing disorder and corresponding characteristics. The titration device generating output data for adjusting the air pressure supplied to the patient as a function of the characteristics of the breathing disorder.

### BACKGROUND

Obstructive sleep apnea/hypopnea syndrome (OSAHS) is a well recognized disorder which may affect as much as 1-5% of the adult population. OSAHS is one of the most common causes of excessive daytime somnolence. OSAHS is most frequent in obese males, and it is the single most frequent reason for referral to sleep disorder clinics.

OSAHS is associated with conditions in which there is anatomic or functional narrowing of the patient's upper airway, and is characterized by an intermittent obstruction of the upper airway during sleep. The obstruction results in a spectrum of respiratory disturbances ranging from the total absence of airflow despite continued respiratory effort (apnea), to significant obstruction with or without reduced airflow (hypopnea, episodes of elevated upper airway resistance, and snoring). Morbidity associated with the syndrome arises from hypoxemia, hypercapnia, bradycardia and sleep disruption associated with the respiratory obstructions and arousals from sleep.

The pathophysiology of OSAHS is not fully worked out. However, it is now well recognized that obstruction of the upper airway during sleep is in part due to the collapsible behavior of the supraglottic segment of the respiratory airway during the negative intraluminal pressure generated by inspiratory effort. The human upper airway during sleep behaves substantially similar to a Starling resistor which by definition limits the flow to a fixed value irrespective of the driving (inspiratory) pressure. Partial or complete airway collapse can occur associated with the loss of airway tone, which is characteristic of the onset of sleep and may be exaggerated with OSAHS.

Since 1981, positive airway pressure (“PAP”) applied by a tightly fitted nasal mask worn during sleep has evolved to become the most effective treatment for this disorder, and is now the standard of care. The availability of this non-invasive form of therapy has resulted in extensive publicity for sleep apnea/hypopnea and increased appearance of large numbers of patients who previously may otherwise avoid medical treatment because of the fear of tracheostomy. Increasing the comfort of the system (e.g., by minimizing the applied nasal pressure) has been a major goal of research aimed at improving patient compliance with therapy.

PAP therapy has become the mainstay of treatment in Obstructive Sleep Disordered Breathing (“OSDB”), which includes Obstructive Sleep Apnea/Hypopnea, Upper Airway Resistance Syndrome, Snoring, exaggerated rises of sleep-induced collapsibility of the upper airway and all conditions in which inappropriate collapsing of a segment of the upper airway causes significant non-physiologic obstruction to airflow. Collapse of a portion of the airway generally occurs whenever pressure in the collapsible portion of the airway becomes sub-atmospheric. Stated another way, collapse occurs when pressure in the airway falls below a “tissue pressure” in the surrounding wall. PAP therapy is directed to maintaining pressure in the collapsible portion of the airway at or above the critical “tissue pressure” at all times. This goal is achieved by raising the airway pressure in the entire respiratory system to a level higher than this critical pressure.

Despite its success, conventional PAP systems have certain. For example, the determination of the appropriate pressure for therapy, referred to as PAP titration, is normally performed in a sleep laboratory where a specific treatment pressure is determined. However, during the first week of treatment the necessary pressure to treat the OSDB may decrease, which results in a prescribed pressure that is too high and may compromise patient compliance. In addition, the patient may assume body positions or sleep stages, other than those occurring in the sleep laboratory that may change the therapeutic pressure. Finally, patients may require periodic retitration following changes in condition, such as weight gain or loss. Retitration of the PAP in the laboratory is usually expensive and is not part of the usual standard of care. Thus, there is a need for a system and method that would provide initial PAP titration and retitration to patients as required during subsequent treatments.

### SUMMARY OF THE INVENTION

The present invention relates to a method and system for automated titration of CPAP. The system may include an air pressure supply providing air pressure to a patient's airways and a sensor detecting input data corresponding to a patient's breathing patterns of a plurality of breaths. The system also includes a titration device which receives and analyzes the input data to determine existence of breathing disorder and corresponding characteristics. The titration device generating output data for adjusting the air pressure supplied to the patient as a function of the characteristics of the breathing disorder

### BRIEF DESCRIPTION OF THE DRAWINGS

The accompanying drawings which are incorporated in and constitute part of the specification, illustrate several embodiments of the invention and, together with the description, serve to explain examples of the present invention. In the drawings:

FIG. 1 shows a waveform of airflow from a sleeping patient in a 30 second epoch when subjected to a substantially constant PAP pressure of 10 cm H₂O;

FIG. 2 shows a waveform of airflow from a sleeping patient in a 30 second epoch when subjected to a substantially constant PAP pressure of 8 cm H₂O;

FIG. 3 shows a waveform of airflow from a sleeping patient in a 30 second epoch when subjected to a substantially constant PAP pressure of 6 cm H₂O;

FIG. 4 shows a waveform of airflow from a sleeping patient in a 30 second epoch when subjected to a substantially constant PAP pressure of 4 cm H₂O;

FIG. 5 shows a waveform of airflow from a sleeping patient in a 30 second epoch when subjected to a substantially constant PAP pressure of 2 cm H₂O;

FIG. 6 shows an exemplary embodiment of a system according to the present invention; and

FIG. 7 shows an exemplary embodiment of a method according to the present invention.

### DETAILED DESCRIPTION

FIGS. 1-5 illustrate waveforms of flow from a PAP generator, obtained during the testing of a patient in sleep studies. In these tests, the patient was wearing a PAP mask connected to an air source, for example, in the manner illustrated in U.S. Pat. No. 5,065,765, the entire disclosure of which is hereby incorporated by reference. Each of these tests illustrates an epoch of 30 seconds, with the vertical lines depicting seconds during the tests. FIGS. 1-5 depict separate sweeps taken from 1 to 2 minutes apart, and with different pressures from the source of air.

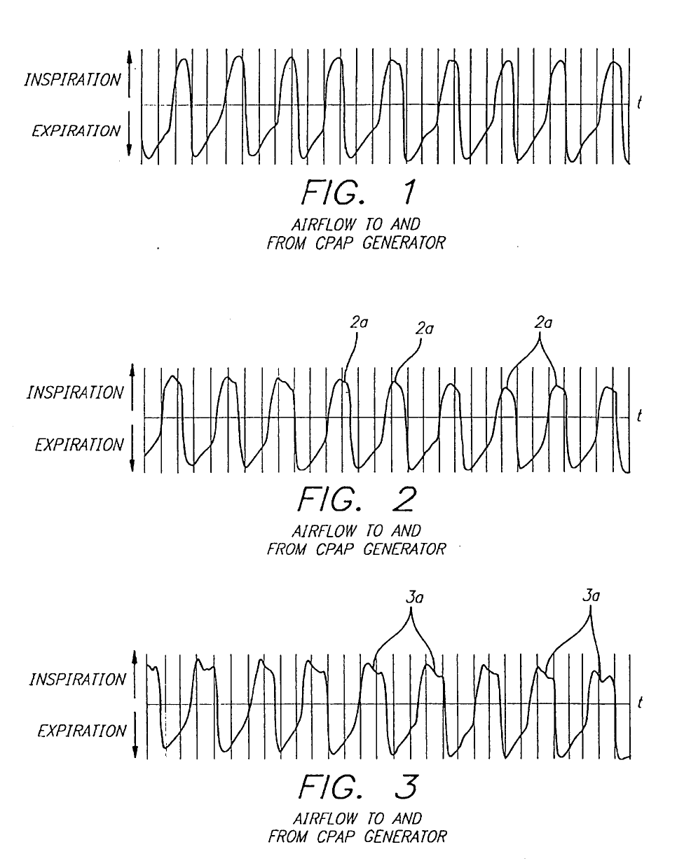

FIG. 1 illustrates a “normal” waveform, in this instance with a Continuous Positive Airway Pressure (“CPAP”) of 10 cm H₂O. Although this description uses a CPAP system to illustrate the system and method according to the present invention, those skilled in the art will understand that this invention is equally useful in conjunction with any variety of PAP systems supplying constant or varying pressure to patients. However, any other pressure identified as corresponding to apnea free respiration may also be used. It is noted that this waveform, at least in the inspiration periods, is substantially sinusoidal. The waveforms of FIGS. 2-5 illustrate that, as the controlled positive pressure is lowered, a predictable index of increasing collapsibility of the airway occurs, prior to the occurrence of frank apnea, periodic breathing or arousal.

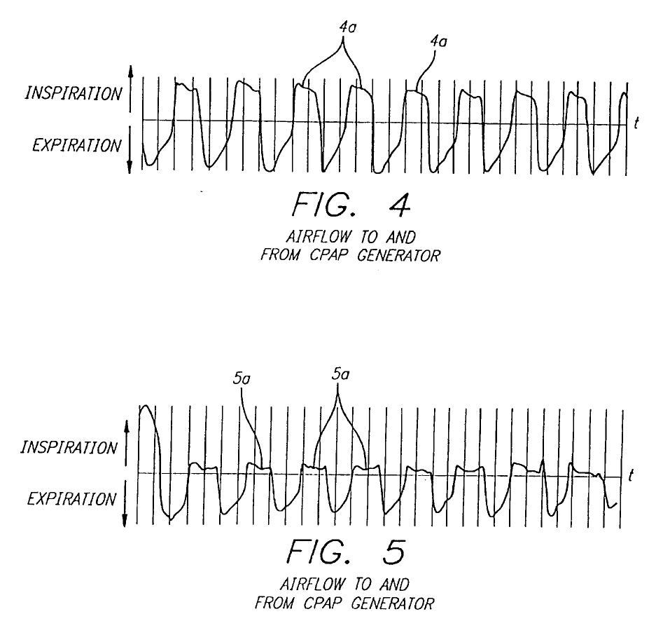

When CPAP pressure is decreased to 8 cm H₂O, as illustrated in FIG. 2, a partial flattening of the inspiratory flow waveform, at region 2a, begins. This flattening becomes more definite when the controlled positive pressure is decreased to 6 cm H₂O, as seen in the region 3a of FIG. 3. The flattening becomes even more pronounced, as seen in the region 4a of FIG. 4, when the controlled positive pressure is reduced to 4 cm H₂O. These reductions in the CPAP pressure from the pressure of apnea free respiration, result in, for example, snoring or other signs of patient airway obstruction. When the CPAP pressure is further reduced to 2 cm H₂O, as illustrated in FIG. 5, inspiratory flow may decrease to a virtually zero level during inspiratory effort, as seen in the region 5a. Shortly after the recording of the waveform of FIG. 5, the patient in the example developed frank apnea and awoke.

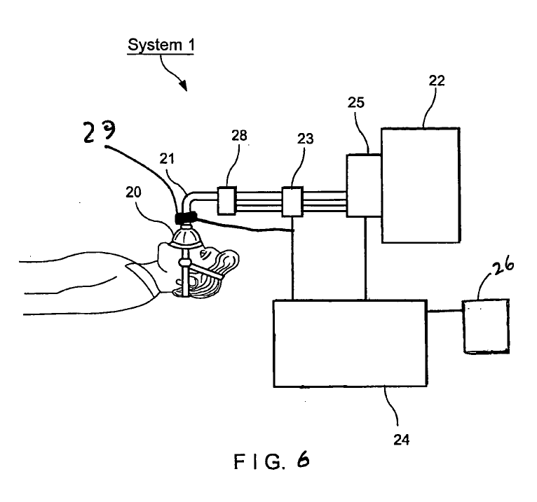

FIG. 6 shows an exemplary embodiment of a system 1 according to the present invention. The system 1 may include a mask 20 that is connected via a tube 21 to receive airflow at a particular pressure from a flow generator 22 or any other suitable airway pressure supply system. The amount of pressure provided to a particular patient varies depending on that patient's particular condition.

The mask 20 covers the patient's nose and/or mouth and conventional flow and/or pressure sensors 23 are coupled to the tube 21 to detect the volume of the airflow to and from the patient and the pressure supplied to the patient by the generator 22. The sensors 23 may be internal or external to the generator 22. Signals corresponding to the airflow and the pressure from the sensors 23 are provided to a processing arrangement 24. The processing arrangement 24 generates pressure control outputs signals to a conventional flow control device 25 that controls the pressure applied to the flow tube 21 by the flow generator 22. Those skilled in the art will understand that, for certain types of flow generators which may be employed as the flow generator 22, the processing arrangement 24 may directly control the flow generator 22, instead of controlling airflow therefrom by manipulating a separate flow control device 25.

The system 1 may also include a venting arrangement 28 which allows for gases exhaled by the patient to be diverted from the incoming air to prevent re-breathing of the exhaled gases. In an alternative exemplary embodiment of the present invention, the system 1 may include a further sensor 29 situated at or near the mask 20. The further sensor 29 is connected to the processing arrangement 24 and provides data regarding the airflow and the pressure in the mask 20 to the processing arrangement 24.

Those skilled in the art will understand that the system 1 may be utilized for the purpose of detecting abnormal respirations and flow limitations in the patient's airway. Alternatively, the system 1 may be utilized for detection of sleeping disorders (e.g., flow limitations), autotitration and treatment of such sleeping disorders.

The system 1 also includes an automatic titration device 26 which provides an initial titration (i.e., determination of an appropriate pressure or an appropriate varying pressure function for a particular patient) as well as subsequent retitrations. The titration device 26 may be a portable device which is attachable (e.g., using convention wired or wireless techniques) to the processing arrangement 24 when it is necessary to obtain appropriate pressure for the PAP therapy or to update previously calculated pressures. Those skilled in the art will understand that the titration device 26 may be attached to any conventional PAP therapy system. Alternatively, the titration device 26 may be built into the system 1 (e.g., the titration device 26 may be combined with the processing arrangement 24).

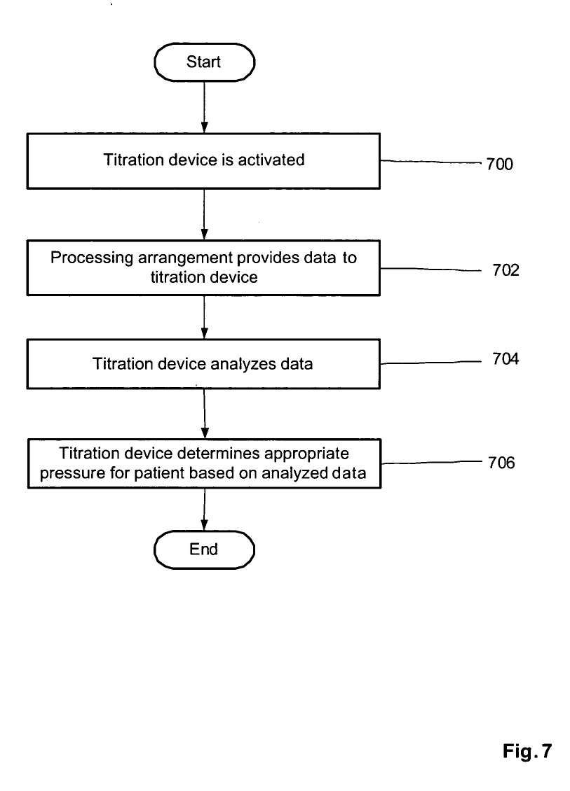

FIG. 7 shows an exemplary method according to the invention for automatic titration to determine an appropriate pressure or varying pressure function for the PAP therapy. In step 700, the titration device 26 is activated, e.g., (a) by powering the titration device 26 if it is a part of the processing arrangement 24 or (b) by connecting the titration device 26, if it is a stand-alone unit, to the processing arrangement 24. Since it may not be necessary to perform titration on a daily basis, the titration device 26 may be activated by the patient or medical personnel initially to obtain appropriate data for calculation of the pressure or pressure function for the PAP therapy. The titration device 26 can be again activated at such times as may be determined are desired to retitrate to ensure the PAP therapy is properly tailored to the patient's current condition. The activation process may be performed immediately prior to initiation of the PAP therapy or may be preset to automatically activate at predetermined points, such as days and/or times.

Once activated, the titration device 26 may remain active for a predetermined period of time. For example, the titration device 26 may remain active for a specific period of time (e.g., a single sleeping cycle of 6-8 hours) or until it is manually deactivated. While active, the titration device 26 may work in the background processing and analyzing data collected by the processing arrangement 24 (step 702) without interfering with the PAP therapy. In particular, the processing arrangement 24 transmits data to the titration device 26 data which includes, among other information, the patient's airflow and the pressure applied to the airways of the patient. Such data may be provided continuously or periodically (e.g., every hour). Alternatively, the titration device 26 may be programmed to update immediately the PAP treatment under predetermined conditions.

The data collected by the titration device 26 may be stored in a database with, for example, data related to each particular patient collected during various titration procedures. Or, collected data may be stored together so that the data from several titration procedures may be accessed and analyzed by the titration device 26 to determine appropriate pressure controls for that patient. For example, the data may be stored on a removable memory arrangement which may be kept by the patient and provided to the titration device 26 each time the titration procedure for this patient is initiated. Alternatively, data for multiple patients may be stored in corresponding files of a single memory arrangement. Those skilled in the art would understand that the single memory arrangement may be a part of the system 1; alternatively, the single memory arrangement may be situated at a remote location that can be accessed via a communications network. (e.g., the Internet, VPN, etc.)

In step 704, the titration device 26 analyzes the collected data. In particular, data relating to patient airflow is utilized to accurately map patient's breathing patterns. The titration device 26 analyzes these breathing patterns to detect abnormal respiratory events and to identify the conditions under which they arise. Abnormal respiratory events that may be identified include apnea, hypopnea and events of elevated upper airway resistance. Apnea is identified by a cessation of respiratory airflow in the patient, where the cessation can last, for example, approximately ten seconds. Hypopnea is identified by a decrease in amplitude of the airflow signal relative to a baseline value, where the decrease can last, for example, approximately ten seconds. Elevations in the resistance of the upper airway may be identified by changes in the shape of the inspiratory airflow contour. The airflow signal from the entire collection period may be analyzed for the presence of sleep disordered breathing events.

In step 706, based on the analysis of respiratory events, the titration device 26 determines, using a predefined algorithm, an appropriate pressure or a varying pressure function to be supplied to the patient. The counts other indexes of respiratory events (e.g., a total time of abnormal respiration, a percentage of abnormal breath, total number of events in general and by type, etc.) that occurred during the previous collection period indicate the efficacy of the pressure administered. When the count or index increases to beyond a preset absolute value or relative value (compared to previous values for that patient) the pressure may be increased for the next CPAP period. If the number of events is below a preset value then the pressure may be decreased for the next predefined time period. In addition, the response to previous pressure decreases may also be incorporated into the pressure determination algorithm. For example, the titration device 26 may determine that a constant pressure supplied to the patient needs to be increased if a number of abnormal events identified reaches a threshold within a specified time period (e.g., when number of apneas, hypopneas or elevated resistance events exceeds the preset limit or increases by a specified amount above the previous values for the patient).

Alternatively, the supplied pressure may need to be decreased or remain unchanged if no abnormal respiratory events are detected or if the number detected is less than the threshold level. If the titration device 26 is used to adjust a variable pressure supplied to a patient, those skilled in the art will understand that, based on the number of abnormal events identified and the circumstances under which they occurred, any number of modifications of the pressure supply function may be initiated. For example, if a pressure supplied to the patient varies substantially sinusoidally, an average value or an amplitude of the pressure may be adjusted.

In the preferred embodiment of the present invention, the titration device 26 may analyze data collected during, e.g, a predetermined time period. For example, the predetermined time period may be a single sleeping cycle such as one night of observation. Alternatively, or in addition, the predetermined time period may be a portion of the single sleeping cycle such as one or two hours of observation. The pressure may be adjusted for the subsequent time period. For example, the pressure may be adjusted once per hour in response to events occurring during the previous hour.

The titration process may then be repeated during the subsequent time period using the adjusted pressure to evaluate the efficacy of the adjusted pressure. Thus, over a several time periods, the titration process may be repeated to enhance the accuracy with which the appropriate pressure is determined. In an alternative embodiment, the titration device 26 may be adapted to continually collect data for the entire duration of the treatment so that the titration process is continuously updated.

As described above, the titration device 26 according to the present invention may be manufactured as a portable stand-alone unit. Such a unit may be easily attached to most conventional therapy systems by positioning the device in the flow path, parallel to the patient and the flow generator 22. If the generator 22 were externally controllable (e.g., by a serial interface), then the titration device 26 may be connected to an external control. Alternatively, a variable pressure valve could be incorporated into the stand-alone unit to control the pressure directly. The valve can mitigate the cost of a therapy system since the patient may rent the titration device 26 only when titration is necessary.

The system 1 may determine appropriate pressures by adjusting pressure only at the beginning of a sleeping cycle and by operating over the course of several sleeping cycles to arrive at a more accurate image of the patient's breathing patterns. For example, some patients may have “good” or “bad” nights which may not be representative of an “average” night for the patient. In contrast, conventional automatic titrating systems may generate immediate feedback responses to the abnormal respiratory events from which they attempt to determine a single therapeutic pressure. Conventional titration systems generally obtain data only during a single sleeping cycle, since multiple visits to sleep clinics, where these systems are located, are unlikely. Furthermore, the more accurate the pressure supplied to a particular patient, the more likely the patient will regularly make use of this PAP therapy.

Another advantage of the present invention is that it may also be used in ongoing treatment of OSDB patients with varying pressure needs. In these cases, the titration device 26 is connected to the PAP therapy system continually so that the pressure supplied may be constantly adjusted by retitration.

It will be apparent to those skilled in the art that various modifications and variations can be made in the structure and the methodology of the present invention, without departing from the spirit or scope of the invention. Thus, it is intended that the present invention cover the modifications and variations of this invention provided they come within the scope of the appended claims and their equivalents.

1. A system, comprising:
an air pressure supply arrangement providing air pressure to a patient's airways;
a sensor detecting input data corresponding to a patient's breathing patterns of a plurality of breaths; and
a titration device receiving and analyzing the input data to determine existence of breathing disorder and corresponding characteristics, the titration device generating output data for adjusting the air pressure supplied to the patient as a function of the characteristics of the breathing disorder.

2. The system according to claim 1, wherein the input data is obtained for at least one time period prior to generating the output data.

3. The system according to claim 2, wherein the at least one time period includes a sleep session that starts when the patient falls asleep and ends when the patient is awakened.

4. The system according to claim 2, wherein the at least one time period includes at least one portion of a sleep session, the sleep session starting when the patient falls asleep and ending when the patient is awakened.

5. The system according to claim 2, wherein the pressure supplied to the patient is adjusted as a function of the output data.

6. The system according to claim 1, wherein the titration device is a portable unit which is removably attached to the air pressure supply arrangement.

7. The system according to claim 1, further comprising:
an arrangement covering at least one of a nose and a mouth of the patient.

8. The system according to claim 7, further comprising:
a tube connected to the arrangement, the air pressure supply arrangement providing a flow of air via the tube to the arrangement.

9. The system according to claim 8, further comprising:
a further sensor coupled to the arrangement, the further sensor adapted to provide a portion of the input data to the titration device related the flow of air in the arrangement.

10. The system according to claim 1, further comprising:
a venting arrangement allowing gases exhaled by the patient to be diverted from incoming air.

11. The system according to claim 1, wherein the sensor is external to the air pressure supply arrangement.

12. The system according to claim 1, wherein the sensor is internal to the air pressure supply arrangement.

13. A method, comprising the steps of:
activating a titration device;
obtaining input data by the titration device from a sensor, the input data corresponding to a patient's breathing patterns;
determining with the titration device existence in the input data one of a breathing disorder and an abnormal flow limitation and corresponding characteristics; and
generating using the titration device an output data as a function of the characteristics for adjusting the pressure provided to the patient.

14. The method according to claim 13, wherein a currently supplied airflow pressure is utilized to generate the output data.

15. The method according to claim 13, wherein the titration device is activated by one of applying power to the titration device and wherein the method further comprising the step of:
connecting the titration device to a Continuous Positive Airway Pressure System.

16. The method according to claim 13, further comprising the step of:
deactivating the titration device after a predetermined time period.

17. The method according to claim 13, wherein the input data is obtained for at least one time period prior to generating the output data.

18. The method according to claim 13, wherein the at least one time period includes at least one sleep session that starts when the patient falls asleep and ends when the patient is awakened.

19. The method according to claim 18, wherein the at least one time period includes at least one portion of a sleep session, the sleep session starting when the patient falls asleep and ending when the patient is awakened.

20. The method according to claim 18, further comprising the step of:
adjusting the pressure supplies to the patient as a function of the control data.

21. The method according to claim 13, further comprising the step of:
storing the input and output data in a memory arrangement.

22. The method according to claim 21, wherein the memory arrangement is portable and removable from the titration device.

23. The method according to claim 13, wherein the breathing disorder includes one or more of apnea, hypopnea and elevation in a resistance of an upper airway of the patient.

24. The method according to claim 13, wherein the input data is obtained until a predetermined event occurs.

25. The method according to claim 24, wherein the predetermined event includes at least one of an index of breathing disorders and a predetermined deviation value of the breathing disorder as compared a predefined value.

26. The method according to claim 25, further comprising the steps of:
generating the output data to decrease the pressure, when the index of the breathing disorders is lower than a predefined value.

27. The method according to claim 25, further comprising the steps of:
generating the output data to increase the pressure, when the index of the breathing disorders is greater than a predefined value

28. A method, comprising the steps of:
coupling to a positive airway pressure supply system a removable diagnostics unit, the positive airway supply system including a source of positive pressure, an airway for supply of the positive pressure to a patient's airway and at least one sensor sensing data corresponding to breathes of the patient, the diagnostics unit including a processor, a memory, an input module receiving input data from the at least one sensor and an output module for outputting control data to the positive airway pressure supply system;
obtaining the input data from the at least one sensor to be stored in the memory;
processing the input data obtained from the at least one sensor using the processor to determine a breathing disorder and corresponding characteristics;
generating the control data for adjusting operation of the positive airway pressure supply system based on the characteristics of the breathing disorder determined by the processor; and
transmitting the control data to the positive airway pressure supply system via the output module.

29. The method according to claim 28, wherein the data is obtained for at least one time period prior to generating the control data.

30. The method according to claim 29, wherein the at least one time period includes at least one sleep session that starts when the patient falls asleep and ends when the patient is awakened.

31. The method according to claim 29, wherein the at least one time period includes at least one portion of a sleep session, the sleep session starting when the patient falls asleep and ending when the patient is awakened.

32. The method according to claim 29, further comprising the step of:
adjusting the pressure supplies to the patient as a function of the control data.

33. A diagnostic device, comprising:
an input module receiving input data from at least one sensor, the input data corresponding to breathing patterns of a patient
a memory storing the input data;
a processor processing the input data to determine a breathing disorder and corresponding characteristics, the processor generating output data for adjusting operation of a positive airway pressure supply system based on the characteristics of the breathing disorder; and
an output module outputting the output data to the system,
wherein the device is a removably coupled the system.

34. The device according to claim 33, wherein the input data is obtained for at least one time period prior to generating the control data.

35. The device according to claim 34, wherein the at least one time period includes at least one sleep session that starts when the patient falls asleep and ends when the patient is awakened.

36. The device according to claim 34, wherein the at least one time period includes at least one portion of a sleep session, the sleep session starting when the patient falls asleep and ending when the patient is awakened

37. The system according to claim 2, wherein the at least one time period is at least one sleep cycle of a patient.

38. A method for titration of an air pressure supplied to a patient's airway, comprising the steps of:
(a) obtaining input data by a titration device from a sensor, the input data corresponding to a patient's breathing patterns;
(b) determining, with the titration device, existence in the input data of one of (i) a breathing disorder and corresponding characteristics thereof and (ii) an abnormal flow limitation and corresponding characteristics thereof;
(c) generating, using the titration device, output data as a function of the characteristics; and
(d) adjusting the air pressure supplied to the patient's airway as a function of the output data.

39. The method according to claim 38, wherein steps (a)-(d) are performed over a first predetermined time period.

40. The method according to claim 39, wherein the first predetermined time period is at least one sleep cycle of the patient, the sleep cycle starts when the patient falls asleep and terminates when the patient is awakened.

41. The method according to claim 38, wherein steps (a)-(d) are repeated over at least one further predetermined time period, the at least one further predetermined time period separated temporally from the first predetermined time period.

42. The method according to claim 41, wherein during the at least one further predetermined time period, further input data is obtained and further output data is generated.

43. The method according to claim 42, wherein a comparison is made of at least one of the further input data and the further output data to at least one of the input data and the output data.

44. The method according to claim 43, wherein the air pressure supplied to the patient is determined as a function of the comparison.
